(19) United States
(12) Patent Application Publication
(10) Pub. No.: US 2012/0052058 A1
(43) Pub. Date: Mar. 1, 2012

(54) METHODS OF MANAGING THE BLOOD COAGULATION AND PHARMACEUTICAL COMPOSITIONS RELATED THERETO

(75) Inventors: Kenichi Tanaka, Atlanta, GA (US); Jerrold H. Levy, Alanta, GA (US); Fania Szlam, Norcross, GA (US)

(73) Assignee: EMORY UNIVERSITY, Atlanta, GA (US)

(21) Appl. No.: 13/219,768

(22) Filed: Aug. 29, 2011

Related U.S. Application Data
(60) Provisional application No. 61/378,107, filed on Aug. 30, 2010.

(51) Int. Cl. A61K 38/10; A61P 7/02; A61K 31/7088; A61K 31/702; A61K 39/395

(52) U.S. Cl. 424/130.1; 514/13.7; 514/54; 514/44 R

(57) ABSTRACT

The disclosure relates to methods of managing blood coagulation and pharmaceutical compositions related thereto. In certain embodiments, the disclosure relates to methods of managing the coagulation of blood in a patient undergoing surgery comprising administering combinations of a direct thrombin inhibitor and a coagulation factor inhibitor. In some embodiments, the disclosure relates to treating or preventing clotting by administering compositions disclosed herein to a subject at risk for, exhibiting symptoms of, or diagnosed with a blood clot.

This application claims priority to U.S. Provisional Application No. 61/378,107 filed 30^{th} of Aug. 2010, hereby incorporated by reference.

### FIELD

The disclosure relates to methods of managing blood coagulation and pharmaceutical compositions related thereto. In certain embodiments, the disclosure relates to methods of managing the coagulation of blood in a patient undergoing surgery comprising administering combinations of a direct thrombin inhibitor and a coagulation factor inhibitor. In some embodiments, the disclosure relates to treating or preventing clotting by administering compositions disclosed herein to a subject at risk for, exhibiting symptoms of, or diagnosed with a blood clot.

### BACKGROUND

Given the central role of thrombosis in the pathobiology of acute ischemic heart disease, anticoagulants have become the foundation of medical treatment for patients presenting with acute coronary syndromes, such as unstable angina, and myocardial infarction and for those undergoing coronary revascularization procedures. Currently available anticoagulants include unfractionated heparin (UFH), the low molecular weight heparins (LMWH), and the direct thrombin inhibitors (DTI). The present paradigm both for anticoagulant use and for continued antithrombotic drug development is to establish a balance between efficacy, which means reducing the risk of ischemic events, and safety, which means minimizing the risk of bleeding. Each of the available agents carries an increased risk of bleeding relative to placebo.

The major adverse event associated with anticoagulant and antithrombotic drugs is bleeding, which can cause permanent disability and death. Generally, cardiovascular clinicians have been willing to trade off an increased risk of bleeding when a drug can reduce the ischemic complications of either the acute coronary syndromes or of coronary revascularization procedures. However, recent data have suggested that bleeding events, particularly those that require blood transfusion, have a significant impact on the outcome and cost of treatment of patients with ACS. Transfusion rates in patients undergoing elective coronary artery bypass graft (CABG) surgery range from 30-60%, and transfusion in these patients is associated with increased short, medium and long-term mortality. Bleeding is also the most frequent and costly complication associated with percutaneous coronary interventions (PCI), with transfusions being performed in 5-10% of patients at an incremental cost of $8000-$12,000. In addition, the frequency of significant bleeding in patients undergoing treatment for ACS is high as well, ranging from 5% to 10% (excluding patients who undergo CABG), with bleeding and transfusion independently associated with a significant increase in short-term mortality. Therefore, despite the continued development of novel antithrombotics, a significant clinical need exists for safer anticoagulant agents.

Reversal of drug activity may be achieved passively by formulation of a drug as an infusible agent with a short half-life with termination of infusion as the means to reverse, or actively via administration of an antidote that can neutralize the activity of the drug. For hospitalized patients with acute ischemic heart disease, the ideal anticoagulant would be deliverable by intravenous or subcutaneous injection, immediately effective, easily dosed so as not to require frequent monitoring and immediately and predictably reversible.

UFH is the only antidote-reversible anticoagulant currently approved for use. However, UFH has significant limitations. First, heparin has complex pharmacokinetics that makes the predictability of its use challenging. Second, the dose predictability of its antidote, protamine, is challenging, and there are serious side effects associated with its use. Finally, heparin can induce thrombocytopenia (HIT) and thrombocytopenia with thrombosis (HITT).

Despite these limitations, heparin remains the most commonly used anticoagulant for hospitalized patients primarily because it is “reversible.” Newer-generation anticoagulants, such as the LMWHs have improved upon the predictability of UFH dosing and do not require lab-based monitoring as part of their routine use. HIT and HITT are observed less frequently with the LMWHs, relative to UFH, but they have not eliminated this risk. Two of the three commercially available DTIs, lepirudin and argatroban, are specifically approved for use in patients who have developed or have a history of HIT. Bivalirudin is approved for use as an anticoagulant during PCI and therefore provides an attractive alternative to UFH in patients who have HIT. However, there are no direct and clear antidotes to reverse the anticoagulant effects of the LMWHs, nor of the DTIs, which presents a particular risk to their use in patients undergoing surgical or percutaneous coronary revascularization procedures. Bleeding in patients treated with LMWH's or DTI's is managed by blood transfusion. Thus, there is a need to identify improved methods.

### SUMMARY

The disclosure relates to methods of managing blood coagulation and pharmaceutical compositions related thereto. In certain embodiments, the disclosure relates to methods of managing the coagulation of blood comprising administering an effective amount of a direct thrombin inhibitor and one or more other activated coagulation factor inhibitor(s) to a subject in need thereof. In a typical embodiment, a direct thrombin inhibitor and a FXa inhibitor are used. In another embodiment, a direct thrombin inhibitor, a FXa inhibitor, and a FIXa inhibitor are used.

In certain embodiments, the direct thrombin inhibitor is bivalirudin, hirudin, lepirudin, desirudin, argatroban, melagatran, or dabigatran and the activated coagulation factor inhibitor is selected from fondaparinux, idraparinux, idrabiotaparinux, bemiparin, certoparin, dalteparin, enoxaparin, nadroparin, parnaparin, reviparin, tinzaparin, apixaban, otamixaban, and rivaroxaban.

In certain embodiments, the activated coagulation factor inhibitor is a contact pathway inhibitor such as a FVIIIa, FIXa, FXa, FXIa, and/or a FXIIa inhibitor. In certain embodiments, the activated coagulation factor inhibitor is an antibody or aptamer such as an anti-FIXa aptamer. In certain embodiments, the method further comprises the step of administering another antithrombotic agent such as aspirin, clopidogrel, and/or cangrelor.

In certain embodiments, the subject is undergoing percutaneous transluminal coronary angioplasty, percutaneous coronary intervention, coronary artery bypass graft, hemodialysis, catheter ablation, or the subject is diagnosed with, exhibiting symptoms of, or at risk of heparin-induced thrombocytopenia/heparin-induced thrombocytopenia thrombosis syndrome, atrial fibrillation, pulmonary embolism, deep vein thrombosis, venous thromboembolism, coronary ischemia, stroke, myocardial infarction, genetic or acquired hypercoagulability. In a typical embodiment, the subject is to undergo any coronary intervention or cardiopulmonary bypass procedure in which heparin anticoagulation may be contraindicated or undesirable.

In certain embodiments, managing the blood coagulation is connected with abnormal thrombus formation, acute myocardial infarction, cardiovascular disorders, unstable angina, thromboembolism, acute vessel closure associated with thrombolytic therapy or percutaneous transluminal coronary angioplasty, transient ischemic attacks, stroke, intermittent claudication, bypass grafting of the coronary or peripheral arteries, vessel luminal narrowing, restenosis post coronary or venous angioplasty, maintenance of vascular access patency in long-term hemodialysis patients, pathologic thrombus formation occurring in the veins of the lower extremities following abdominal, knee or hip surgery, pathologic thrombus formation occurring in the veins of the lower extremities following abdominal, knee and hip surgery, a risk of pulmonary thromboembolism, or disseminated systemic intravascular coagulatopathy occurring in vascular systems during septic shock, viral infections or cancer, or reducing an inflammatory response, fibrinolysis, or treatment of coronary heart disease, myocardial infarction, angina pectoris, vascular restenosis, adult respiratory distress syndrome, multi-organ failure and disseminated intravascular clotting disorder, deep vein or proximal vein thrombosis.

In certain embodiments, the subject is a human, livestock or domestic pet.

In certain embodiments, administration is provided under conditions such that the patient plasma concentrations of the direct thrombin inhibitor are less than 5, 1, 0.5, 0.1, or 0.01 micrograms per milliliter. In certain embodiments, administration is provided under conditions such that the patient plasma concentrations of the activated coagulation factor inhibitor are less than 5, 1, 0.5, 0.1, or 0.01 micrograms per milliliter.

In certain embodiments, the disclosure relates to method of administering bivalirudin and fondaparinux to a subject. In certain embodiments, bivalirudin is administer at less than 0.2, 0.1 or 0.05 mg/kg IV bolus followed by less than 0.5, 0.2, 0.1, or 0.05 mg/kg/h and fondaparinux is administered once at less than 2.5, 1.0, 0.5, 0.1, 0.05, or 0.01 mg.

In certain embodiments, the disclosure relates to administering bivalirudin in patients undergoing surgery in an IV bolus dose of less than 0.75, 0.50, 0.20, 0.10, or 0.05 mg/kg in combination with an FXa or FIXa inhibitor or other contact pathway inhibitor. In certain embodiments, the initial dose is followed by infusion at a rate of about 1.75, 1.00, 0.50, 0.20, 0.10, or 0.05 mg/kg/h for the duration of the procedure. Continuation of the infusion following surgery for up to 1, 2, 3, 4, 5, or 6, hours (typically 4 hours) post-procedure is optional. After that an additional IV infusion of bivalirudin may be initiated at a rate of about 0.20 mg/kg/h, for up to day or more, as needed. Bivalirudin is typically administered with aspirin.

In some embodiments, the disclosure relates to pharmaceutical compositions comprising a direct thrombin inhibitor and an activated coagulation factor inhibitor. In certain embodiments, the disclosure relates to managing blood coagulation by administering the pharmaceutical composition to a subject in need thereof. In certain embodiment, the disclosure relates to the use of a direct thrombin inhibitor and an activated coagulation factor inhibitor in the production of a medicament for the management of blood coagulation.

### BRIEF DESCRIPTION OF THE FIGURES

FIG. 1 illustrates contact pathways.

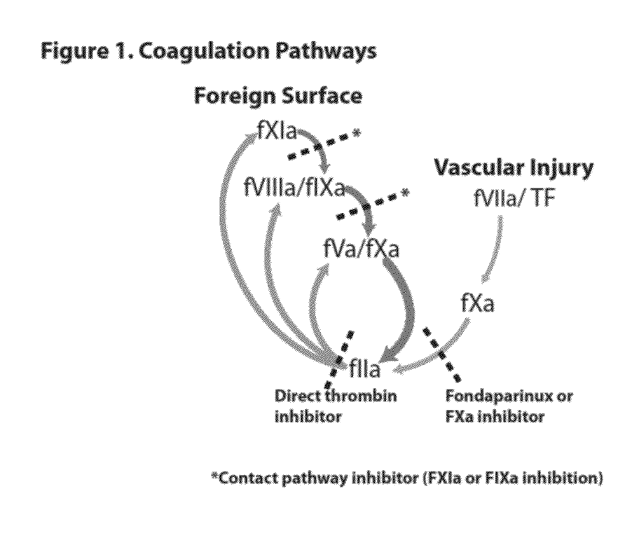

FIG. 2A shows data on the dose response of bivalirudin on thrombin generation in normal plasma.

FIG. 2B shows data from studies of bivalirudin and fondaparinux in normal plasma.

FIG. 2C shows data from studies of bivalirudin and fondaparinux on thrombin generation in Factor IX deficient plasma.

### DETAILED DISCUSSION

### Blood Coagulation Pathways

The blood clothing system is a proteolytic cascade. Each enzyme of the pathway is present in the plasma as a zymogen, in other words in an inactive form, which on activation undergoes proteolytic cleavage to release the active factor form the precursor molecule. The ultimate goal is to produce thrombin. Thrombin converts fibrinogen into fibrin, which forms a clot.

The generation of thrombin is initiated in a vascular injury (i.e., wound) by tissue factor (extrinsic) pathway, which subsequently involves intrinsic pathway to augment thrombin formation. The intrinsic pathway is also called the contact pathway because it can be alternatively initiated by the foreign materials including certain glass, plastics, and other materials used in medical devices (e.g., dialysis circuits). As used here, the term “Factor” followed by a roman numeral refers to the biological component involved in the pathway. If the roman numeral is followed by an “a” such as in “FXa” this refers to the activated X factor.

The contact pathway is typically activated when blood comes into contact with foreign material exposed as a result of medical intervention such as hemodialysis or extracorporeal circulation (i.e., heart-lung machine). The Hageman factor (factor XII), factor XI, prekallikrein, and high molecular weight kininogen (HMWK) are involved in this pathway of activation. The first step is the binding of Hageman factor to a sub-endothelial surface exposed by an injury. A complex of prekallikrein and HMWK also interacts with the exposed surface in close proximity to the bound factor XII, which becomes activated. During activation, the single chain protein of the native Hageman factor is cleaved into two chains that remain linked by a disulphide bond. The light chain contains the active site and the molecule is referred to as activated Hageman factor (factor XIIa).

Activated Hageman factor in turn activates prekallikrein. The kallikrein produced can then also cleave factor XII, and a further amplification mechanism is triggered. The activated factor XIIa remains in close contact with the activating surface, such that it can activate factor XI. Also involved at this stage is HMWK, which binds to factor XI and facilitates the activation process. Activated factors XIa, XIIa, and kallikrein are serine proteases.

Eventually the contact pathway activates factor X. Factor X is the first molecule of the common pathway and is activated by a complex of molecules containing activated factor IX (FIXa), factor VIII, calcium, and phospholipids which are on the platelet surface, where this reaction usually takes place. Factor VIII is activated by thrombin, and it facilitates the activation of factor X by FIXa.

### Thrombin Inhibitors

Direct thrombin inhibitors (DTIs) act as anticoagulants by directly inhibiting the enzyme thrombin. Bivalent DTIs (hirudin and analogs) bind both to the active site and exosite 1, while univalent DTIs bind to the active site. Examples of direct thrombin inhibitors include hirudin, Bivalirudin, lepirudin, desirudin, argatroban, dabigatran, melagatran, and ximelagatran. Bivalirudin, also known as Hirulog-8, is a synthetic congener of the naturally occurring thrombin peptide inhibitor hirudin. Hirudin consists of 65 amino acids, although shorter peptide segments have proven to be effective as thrombin inhibitors. U.S. Pat. No. 5,196,404 discloses bivalirudin among these shorter peptides that demonstrate an anticoagulant activity.

Bivalirudin has the chemical name of D-Phenylalanyl-L-Prolyl-L-Arginyl-L-Prolyl-Glycyl-Glycyl-Glycyl-Glycyl-L-Asparagyl-Glycyl-L-Aspartyl-L-Phenylalanyl-L-Glutamyl-L-Glutamyl-L-Isoleuc-yl-L-Prolyl-L-Glutamyl-L-Glutamyl-L-Tyrosyl-L-Leucine trifluoroacetate (salt) hydrate. Bivalirudin is made up of the amino acid sequence: (D-Phe)-Pro-Arg-Pro-Gly-Gly-Gly-Gly-Asn-Gly-Asp-Phe-Glu-Glu-Ile-Pro-Glu-G-lu-Tyr-Leu (SEQ ID NO: 1). Methods for the synthesis of bivalirudin may include, but are not limited to, solid-phase peptide synthesis, solution-phase peptide synthesis, or a combination of solid-phase and solution-phase procedures (e.g. as provided in U.S. Pat. No. 5,196,404 and PCT Patent Application WO 91/02750). The term “bivalirudin” refers to the peptide described above or any salt thereof.

In some embodiments the disclosure relates to co-administration and pharmaceutical compositions having a direct thrombin inhibitor and a factor Xa (FXa) inhibitor. FXa inhibitors include low molecular weight heparins, such as bemiparin, certoparin, dalteparin, enoxaparin, nadroparin, parnaparin, reviparin, tinzaparin; oligosaccharides such as fondaparinux, idraparinux, and idrabiotaparinux; xabans such as apixaban, otamixaban, and rivaroxaban. Fondaparinux sodium is methyl O-2-deoxy-6-O-sulfo-2-(sulfoamino)-α-D-glucopyranosyl-(1→4)-O-β-D-glucopyra-nuronosyl-(1→4)-O-2-deoxy-3,6-di-O-sulfo-2-(sulfoamino)-α-D-glucopyranosyl-(1→4)—O-2-O-sulfo-α-L-idopyranuronosyl-(1→4)-2-deoxy-6-O-sulfo-2-(sulfoamino)-α-D-glucopyranoside, decasodium salt. It mediates its effects indirectly through antithrombin III, but unlike heparin, it is selective for factor Xa. The term “fondaparinux” refers to the polysaccharide described above or any salt thereof. Heparinase I and II produce a concentration-dependent inhibition of the in vivo antithrombotic activity of fondaparinux. In certain embodiments, the disclosure relates to methods that further comprise the step of adding an antidote to eliminate, counteract, diminish, or reverse the effects of any of the agents disclosed herein in a subject in need thereof such as in the case of heparinase I or II for fondaparinux. Idrabiotaparinux is a synthetic anticoagulant that links idraparinux, an indirect Factor Xa inhibitor, and biotin. Intravenous avidin reverses the anti-Factor Xa activity of idrabiotaparinux. Typical antidotes include, but are not limited to, an aptamer, the complimentary strand of an aptamer, an avidin or streptavidin for a biotinylate molecule or a natural or artificially developed antibody for an epitope.

### Aptamers

In certain embodiments, aptamers are contemplated as activated coagulation factor inhibitors. Oligonucleotides can be developed to target a blood factor or activated factor of the coagulation pathways inhibiting activity. SELEX (“Systematic Evolution of Ligands by Exponential Enrichment”) is a combinatorial chemistry technique for producing oligonucleotides of either single-stranded DNA or RNA that specifically bind to a target. Standard details on generating aptamers can be found in U.S. Pat. No. 5,475,096, and U.S. Pat. No. 5,270,163 (see also WO 91/19813).

The SELEX process provides a class of products which are referred to as nucleic acid ligands or aptamers, each having a unique sequence, and which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule.

The basic SELEX method has been modified to achieve a number of specific objectives. For example, U.S. Pat. No. 5,707,796 describes the use of the SELEX process in conjunction with gel electrophoresis to select nucleic acid molecules with specific structural characteristics, such as bent DNA. U.S. Pat. No. 5,763,177 and U.S. Pat. No. 6,011,577 describe a SELEX based method for selecting nucleic acid ligands containing photoreactive groups capable of binding and/or photocrosslinking to and/or photoinactivating a target molecule. U.S. Pat. No. 5,580,737 describes a method for identifying highly specific nucleic acid ligands able to discriminate between closely related molecules, which can be non-peptidic, termed Counter-SELEX. U.S. Pat. No. 5,567,588 describes a SELEX-based method which achieves efficient partitioning between oligonucleotides having high and low affinity for a target molecule.

The SELEX method encompasses the identification of high-affinity nucleic acid ligands containing modified nucleotides conferring improved characteristics on the ligand, such as improved in vivo stability or improved delivery characteristics. Examples include U.S. Pat. No. 5,660,985 and U.S. Pat. No. 5,580,737.

The SELEX method encompasses combining selected oligonucleotides with other selected oligonucleotides and non-oligonucleotide functional units as described in U.S. Pat. No. 5,637,459 and U.S. Pat. No. 5,683,867. These applications allow the combination of the broad array of shapes and other properties, and the efficient amplification and replication properties, of oligonucleotides with the desirable properties of other molecules.

A special form of aptamers are the so-called spiegelmers, which are characterized essentially by being assembled at least partially, preferably completely, from the non-natural L-nucleotides. Methods for the preparation of such spiegelmers are described in WO/1998/008856.

Once elucidated, an aptamer's nucleotide sequence can be used to design an active control agent in the form of an oligonucleotide complementary to the nucleotide sequence of the aptamer. An aptamer and its active control agent bind via nucleotide base pairing, triggering a structural change in the aptamer that causes its release from the target protein, negating the aptamer's therapeutic effect. Based upon the dose of active control agent administered, the activity of an aptamer can be neutralized, or titrated to intermediate levels in a tightly controlled manner. The REG1 system (Regado Biosciences) is a reversible anti-FIXa aptamer paired with its antidote.

The drug-modulator technology has been applied to the discovery of aptamer based, anticoagulation systems such as REG1. The active component of REG1, RB006 is provided in U.S. Published Application No 20090163437. RB006 elicits an anticoagulant effect by blocking the FVIIIa/FIXa-catalyzed conversion of FX to FXa. RB006 is a modified RNA aptamer, 31 nucleotides in length, which is moderately stabilized against endonuclease degradation by the presence of 2′-fluoro and 2′-O-methyl sugar-containing residues, and stabilized against exonuclease degradation by a 3′ inverted deoxythymidine cap. The nucleic acid portion of the aptamer is conjugated to a 40-kilodalton polyethylene glycol (PEG) carrier to enhance its blood half-life.

### Antibodies

In some embodiments, the disclosure relates to the co-administration and pharmaceutical compositions having a direct thrombin inhibitor and a contact pathway inhibitor wherein the inhibitor is a humanized coagulation factor antibody such as anti-FXI similar to those disclosed by Gruber & Hanson (2003) Blood 102(3): 953-955.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See, e.g., U.S. Pat. No. 4,816,567 and U.S. Pat. No. 4,816,397. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

### Antithrombotics

In some embodiments, the disclosure relates to compositions and methods of using a direct thrombin inhibitor and/or an activated coagulation factor inhibitor (typically bivalirudin and fondaparinux) in combination with other antithrombotics (thrombolytics, anticoagulants and antiplatelet drugs) such as aspirin, heparin, heparin sulfate, or danaparoid sodium. As used herein, the term “combination with” when used to describe administration with an additional treatment means that the agent may be administered prior to, together with, or after the additional treatment, or a combination thereof. In certain embodiments, 100-500 mg of daily aspirin, typically 300-350 mg, is administered in combination with a direct thrombin inhibitor and/or an activated coagulation factor inhibitor.

In certain embodiments, the antiplatelet drugs are glycoprotein IIb/IIIa inhibitor such as abciximab, eptifibatide, and tirofiban; ADP receptor/P2Y12 inhibitors such as thienopyridines (clopidogrel, prasugrel, ticlopidine) and ticagrelor; prostaglandin analogues (PGI2) such as beraprost, prostacyclin, iloprost, and treprostinil; COX inhibitors such as acetylsalicylic acid/aspirin, aloxiprin, carbasalate calcium, indobufen, and triflusal; thromboxane synthase inhibitors such as dipyridamole, picotamide; receptor antagonist such as terutroban; phosphodiesterase inhibitors such as cilostazol, dipyridamole, triflusal or others such as cloricromen and ditazole.

In certain embodiments, the antithrombotics are vitamin K antagonists such as coumarins: acenocoumarol, coumatetralyl, dicoumarol, ethyl biscoumacetate, phenprocoumon and warfarin; 1,3-indandiones such as clorindione, diphenadione, and phenindione or others such as tioclomarol.

In certain embodiments, the antithrombotics are defibrotide, ramatroban, antithrombin III, protein C (drotrecogin alfa).

In certain embodiments, the antithrombotics are thrombolytic drugs and/or fibrinolytics plasminogen activators such as r-tPA (alteplase, reteplase, tenecteplase), UPA (urokinase, saruplase), streptokinase, anistreplase, and monteplase.

In certain embodiments, the antithrombotics are other serine endopeptidases such as ancrod and fibrinolysin or others such as brinase.

### Pharmaceutical Formulations

In certain embodiments, the disclosure relates to pharmaceutical formulations comprising a DTI, an activated coagulation factor inhibitor, and optionally additional antithrombotic agents. Typically the DTI is bivalirudin, and method for formulating solutions of bivalirudin are described in U.S. Pat. No. 7,598,343 and may be applied for other DTIs, activated coagulation factor inhibitors, and combination of compounds described herein.

In the compounding process of various embodiments of the present disclosure, compound(s) may be dissolved in a solvent to form a compound(s) solution. Compound(s) may be commercially purchased or synthesized by various procedures. The concentration of compound(s) in the solvent may be between about 0.010 g/mL and about 1 g/mL, or between about 0.050 g/mL and about 0.1 g/mL. Solvents may include aqueous and non-aqueous liquids, including but not limited to, mono- and di-alcohols such as methanol, ethanol, isopropyl alcohol, and propylene glycol; polyhydric alcohols such as glycerol and polyethylene glycol; buffers; and water.

The solvent may comprise carriers such as sugars. For example, the sugar may be a monosaccharide such as glucose or fructose; a disaccharide such as sucrose, maltose, or trehalose; an oligosaccharide; or a polysaccharide. Alternatively, the sugar may be a sugar alcohol, such as sorbitol or mannitol. The quantity of carrier in the solvent may be adjusted to provide a pharmaceutical batch or pharmaceutical formulation preferably having a ratio of the carrier to the active ingredient(s) of between about 5:1 and about 1:10, or between about 1:1 and about 1:4, or more preferably about 1:2.

Compound(s) can be dissolved in the solvent by methods known in the art, preferably by adding the compound(s) to the solvent. For example, compound(s) may be added to the solvent rapidly, slowly, in portions, at a constant rate, at a variable rate, or a combination thereof. A mixing device known in the art may be used to dissolve compound(s). Examples of mixing devices may include, but are not limited to, a paddle mixer, magnetic stirrer, shaker, re-circulating pump, homogenizer, and any combination thereof. The mixing device may be applied at a mixing rate between about 100 and about 2000 rpm, or between about 300 and about 1500 rpm. The solution resulting from dissolving the compound(s) in the solvent is referred to here as the “compound(s) solution” or alternatively the “first solution.”

The compounding process may comprise mixing a pH-adjusting solution with the compound(s) solution to form a compounding solution. The pH-adjusting solution may be prepared before, after, or simultaneously with, the compound(s) solution.

The pH-adjusting solution may comprise a base dissolved in a solvent, wherein the solvent is referred to here as the “pH-adjusting solution solvent.” In other words, the solution resulting from the combination of the base with the pH-adjusting solution solvent is referred to here as the “pH-adjusting solution.” The pH-adjusting solution may also comprise a neat base such as pyridine or a volatilizable base such as ammonium carbonate.

The base may be an organic base or an inorganic base. The terms “inorganic base” and “organic base,” as used herein, refer to compounds that react with an acid to form a salt; compounds that produce hydroxide ions in an aqueous solution (Arrhenius bases); molecules or ions that capture hydrogen ions (Bronsted-Lowry bases); and/or molecules or ions that donate an electron pair to form a chemical bond (Lewis bases). In certain processes, the inorganic or organic base may be an alkaline carbonate, an alkaline bicarbonate, an alkaline earth metal carbonate, an alkaline hydroxide, an alkaline earth metal hydroxide, an amine, or a phosphine. For example, the inorganic or organic base may be an alkaline hydroxide such as lithium hydroxide, potassium hydroxide, cesium hydroxide, or sodium hydroxide; an alkaline carbonate such as calcium carbonate or sodium carbonate; or an alkaline bicarbonate such as sodium bicarbonate.

Solvents may include aqueous and non-aqueous liquids, including but not limited to, mono- and di-alcohols such as methanol, ethanol, isopropyl alcohol, and propylene glycol; polyhydric alcohols such as glycerol and polyethylene glycol; buffers; and water. The pH-adjusting solution solvent may comprise carriers such as dissolved sugars. For instance, the sugar may be a monosaccharide such as glucose or fructose; a disaccharide such as sucrose, maltose, or trehalose; an oligosaccharide; or a polysaccharide. The sugar may also be a sugar alcohol, such as sorbitol or mannitol. The quantity of the carrier in the pH-adjusting solution solvent may be adjusted to provide the final product as described above.

The base is mixed or dissolved in the pH-adjusting solution solvent. The mixing or dissolution can be performed by methods known in the art. For instance, the base may be added to the pH-adjusting solution solvent rapidly, slowly, in portions, at a constant rate, at a variable rate, or a combination thereof. Also, a mixing device known in the art may be used to mix the base and the pH-adjusting solution solvent. Examples of mixing devices may include, but are not limited to, a paddle mixer, magnetic stirrer, shaker, re-circulating pump, homogenizer, and any combination thereof. The mixing device may be applied at a mixing rate between about 100 and about 1500 rpm, or between about 300 and about 1200 rpm. The base is added/mixed with the pH-adjusting solution solvent in a quantity that will result in a pH-adjusting solution that is characterized as being between about 0.01 N and about 5 N, or between about 0.1 N and 1 N.

The pH-adjusting solution may then be mixed with the compound(s) solution. This mixing may occur by adding the pH-adjusting solution to the compound(s) solution. Alternatively, the compound(s) solution may be added to the pH-adjusting solution, or the pH-adjusting solution and the compound(s) solution may be added simultaneously (into a separate vessel), or there may be a combination of these addition methods thereof. It is important during the adding or mixing of the pH-adjusting solution and the compound(s) solution that pH is controlled. See below. The solution resulting from mixing the pH-adjusting solution and the compound(s) solution is referred to here as the “compounding solution,” or the “second solution.” The compounding solution or the second solution can refer to the compound(s) solution during or after the pH-adjusting solution is added, or can refer to the pH-adjusting solution during or after the compound(s) solution is added, or can refer to the resulting solution formed during or after both the pH-adjusting solution and the compound(s) solution are added together.

The mixing of the pH-adjusting solution and the compound(s) solution may occur under controlled conditions. For example, temperature may be controlled by means known in the art, such as by mixing the pH-adjusting solution and the compound(s) solution in a vessel inside a cooling jacket. The temperature may be set between about 1 degree C. and about 25 degree C., or between about 2 degree C. and about 10 degree C. In some instances, the temperature may exceed 25 degree C. for limited periods of time. Also, the mixing of the pH-adjusting solution and the compound(s) solution may occur under controlled conditions such as under nitrogen, etc.

The pH-adjusting solution will be mixed with the compound(s) solution to form the compounding solution. Efficient mixing of the pH-adjusting solution with the compound(s) solution will minimize levels of impurities in the compounding solution.

The isoelectric point of the compound(s) will need to be considered for efficient mixing. For example, bivalirudin has an isoelectric point of about 3.6. As an aqueous bivalirudin solution, without any other components, has a pH of between about 2.5 and about 2.8, and the compounding solution is adjusted to a final pH of between about 5.1 and about 5.5, a portion of bivalirudin precipitates out during the addition of the pH-adjusting solution. For example, if the pH-adjusting solution is introduced without efficient mixing, a dense precipitate may form. Efficient mixing reduces the generation of “hot spots” or high or low levels of pH in the compounding solution while the pH-adjusting solution and the compound(s) solution are being added/mixed. Thus, efficient mixing may control the overall pH level of the compounding solution to a level not exceeding about 8, or a level not exceeding about 7, or a level not exceeding about 6, or even a level not exceeding about 5.5.

Optionally, once the compounding solution is formed, the pH or the final volume of the compounding solution may be adjusted to a specified level before removal of the solvent (see below). The pH or volume can be adjusted using methods known in the art, for instance, the addition of a pH-adjusting solution as described above.

The compounding solution may also be sterilized before the removal of solvent. The compounding solution may undergo aseptic filtration using, for example, a disposable membrane filter, to sterilize the compounding solution. Furthermore, following sterilization, the compounding solution may be aliquotted into containers such as vials, bottles, ampoules, syringes, etc.

The compounding process of various embodiments of the disclosure may comprise removing solvents from the compounding solution in order to produce a pharmaceutical batch(es) or pharmaceutical formulation(s).

Removal of the solvent from the compounding solution may be achieved through lyophilization, which comprises freezing the compounding solution and then reducing the surrounding pressure to allow the frozen solvent/moisture in the material to sublime directly from a solid phase to a gas phase such as provided in U.S. Pat. Nos. 7,351,431, and 6,821,515. The solvent may also be removed from the compounding solution through other techniques such as spray drying and spray-freeze drying, vacuum drying, super critical fluid processing, air drying, or other forms of evaporative drying.

### EXPERIMENTAL

### Example

### Combining Agents so that the Doses May be Decreased

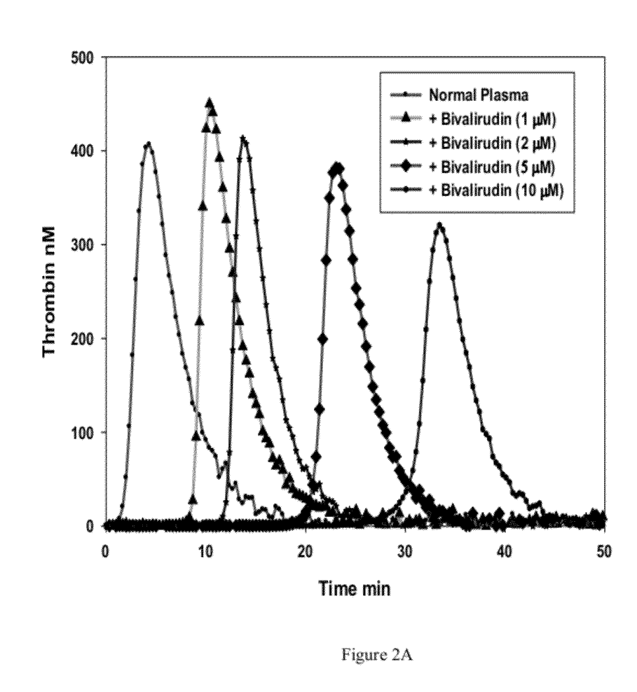

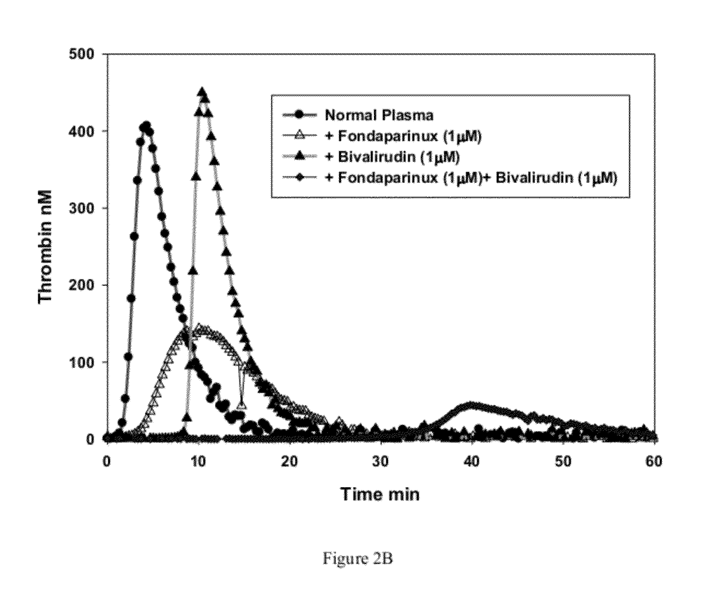

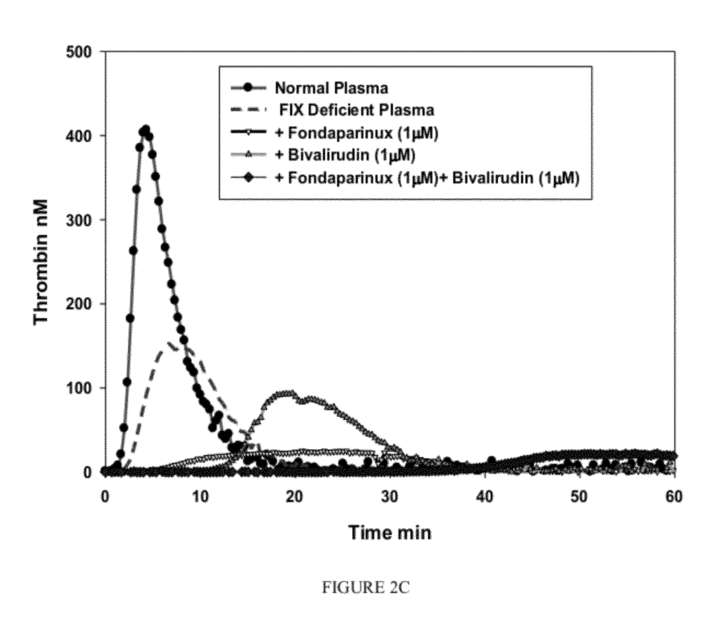

When a FXa inhibitor, fondaparinux is combined with bivalirudin, thrombin generation, as exemplified by decrease in thrombin peak and increase in lag time, is suppressed better than with bivalirudin alone when using procedures as provided in Tanaka & Szlam et al. Anesth Analg 105(4): 933-939 (2007) (or as appropriately modified). See FIG. 2A-2B. Further, when the thrombin generation experiments were repeated in FIX deficient plasma in which contact pathway is inhibited, the effect of fondaparinux/bivalirudin combination was further improved (FIG. 2C). A full anticoagulation comparable to heparin can be achieved without requiring high plasma concentrations of bivalirudin (15-20 μg/ml). Peptides degrade in vivo by proteases. Since bivalirudin is a peptide, as are other DTIs, using lower doses will increase the clearance time and reduce the risk of overdose and bleeding. Termination of infusion may be used as a means to reverse activity.

1. A method of managing the coagulation of blood comprising administering a direct thrombin inhibitor and activated coagulation factor inhibitor to a subject in need thereof.

2. The method of claim 1, wherein the direct thrombin inhibitor is bivalirudin, hirudin, lepirudin, desirudin, argatroban, melagatran, or dabigatran.

3. The method of claim 1, wherein the activated coagulation factor inhibitor is a contact pathway inhibitor.

4. The method of claim 3, wherein the activated coagulation factor inhibitor is selected from a FVIIIa, FIXa, FXa, FXIa, and a FXIIa inhibitor.

5. The method of claim 1, wherein the activated coagulation factor inhibitor is selected from fondaparinux, idraparinux, idrabiotaparinux, bemiparin, certoparin, dalteparin, enoxaparin, nadroparin, parnaparin, reviparin, tinzaparin, apixaban, otamixaban, and rivaroxaban.

6. The method of claim 1, wherein the activated coagulation factor inhibitor is an antibody or aptamer.

7. The method of claim 1, wherein the activated coagulation factor inhibitor is an anti-FIXa aptamer.

8. The method of claim 7, wherein the anti-FIXa aptamer is REG1.

9. The method of claim 1 further comprising the step of administering another antithrombotic agent.

10. The method of claim 1, wherein administration is provided under conditions such that the patient plasma concentrations of the direct thrombin inhibitor are less than 5, 1, 0.5, 0.1, or 0.01 micrograms per milliliter.

11. The method of claim 1, wherein administration is provided under conditions such that the patient plasma concentrations of the activated coagulation factor inhibitor are less than 5, 1, 0.5, 0.1, or 0.01 micrograms per milliliter.

12. The method of claim 1, wherein the subject is undergoing percutaneous transluminal coronary angioplasty, percutaneous coronary intervention, coronary artery bypass graft, hemodialysis, catheter ablation or diagnosed with, exhibiting symptoms of, or at risk of, heparin-induced thrombocytopenia/heparin-induced thrombocytopenia thrombosis syndrome, atrial fibrillation, pulmonary embolism, deep vein thrombosis, or venous thromboembolism, coronary ischemia, stroke, myocardial infarction, genetic or acquired hypercoagulability.

13. A method of managing the coagulation of blood comprising administering bivalirudin and fondaparinux to a subject in need thereof.

14. The method of claim 13, wherein bivalirudin is administer at less than 0.2, 0.1 or 0.05 mg/kg IV bolus followed by less than 0.5, 0.2, 0.1, or 0.05 mg/kg/h and fondaparinux is administered once at less than 2.5, 1.0, 0.5, 0.1, 0.05, or 0.01 mg.

15. A pharmaceutical composition comprising a direct thrombin inhibitor and an activated coagulation factor inhibitor.


SEQUENCE LISTING (1 sequence)

SEQ ID NO: 1 - protein, 61 aa, Artificial Sequence
  Bivalirudin
DPHECCCARGCCCGLYGLYGLYGLYASNGLYASPPHEGAAGAAILECCCGAAGAATYRTTA